(19) United States
(12) Patent Application Publication
(10) Pub. No.: US 2010/0098758 A1
(43) Pub. Date: Apr. 22, 2010

(54) Abuse-Resistant Dosage Form

(75) Inventors: Johannes BARTHOLOMÄUS, Aachen (DE); Klaus-Dieter Langner, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: GRUENENTHAL GmbH, Aachen (DE)

(21) Appl. No.: 12/646,326

(22) Filed: Dec. 23, 2009

Related U.S. Application Data
(62) Division of application No. 11/113,055, filed on Apr. 25, 2005, now abandoned.
Application No. 11/113,055 is a continuation of application No. PCT/EP2003/011789, filed on Oct. 24, 2003.

(30) Foreign Application Priority Data
Oct. 25, 2002 (DE) 102 50 087.8

(51) Int. Cl. A61K 9/24; A61K 31/435; A61K 36/00; A61K 31/4375

(52) U.S. Cl. 424/472; 514/284; 424/773; 514/294

(57) ABSTRACT

A solid pharmaceutical dosage form that is safeguarded against abuse, comprising at least one active substance that is susceptible to abuse and at least one emetic that is spatially separate from the at least one active substance. The active substance or substances are present in the form of at least one sub-unit (a), and the at least one emetic is present in the form of at least one sub-unit (b), and the emetic from sub-unit (b) is not activated in the body if the dosage form has been correctly administered as prescribed.

### CROSS REFERENCE TO RELATED APPLICATIONS

This application is a division of co-pending application Ser. No. 11/113,055, filed Apr. 25, 2005 and now abandoned, which in turn was a continuation of international patent application no. PCT/EP2003/011789, filed Oct. 24, 2003 designating the United States of America, and published in German on May 6, 2004 as WO 2004/037230, the entire disclosures of which are incorporated herein by reference. Priority is claimed based on Federal Republic of Germany patent application no. DE 102 50 087.8, filed Oct. 25, 2002.

### BACKGROUND OF THE INVENTION

The present invention relates to an abuse-resistant solid dosage form comprising at least one active ingredient with potential for abuse and at least one emetic spatially separate therefrom, wherein the active ingredient or active ingredients is/are present in at least one subunit (a) and the emetic is present in at least one subunit (b) and the emetic from subunit (b) does not take effect in the body if the dosage form is correctly administered.

Many pharmaceutical active ingredients, in addition to having excellent activity in their appropriate application, also have potential for abuse, i.e. they can be used by an abuser to bring about effects other than the medical ones intended. Opiates, for example, which are highly active in combating severe to very severe pain, are frequently used by abusers to achieve a state of narcosis or euphoria.

Oral dosage forms which contain such active ingredients with potential for abuse do not usually give rise to the result desired by the abuser, even when taken in an abusively large quantity, because blood levels of the active ingredients increase only slowly. In order nevertheless to enable abuse, the corresponding dosage forms are comminuted, for example ground, by the abuser and administered, for example, by sniffing nasally. In another form of abuse, the active ingredient is extracted from the powder obtained by comminution of the dosage form using a preferably aqueous liquid and the resultant solution, optionally after being filtered through cotton wool or cellulose wadding, is administered parenterally, in particular intravenously. These forms of administration give rise to an accelerated rise in levels of the active ingredient, relative to oral administration, providing the abuser with the desired result.

### SUMMARY OF THE INVENTION

The object of the present invention was therefore to provide a dosage form for active ingredients with potential for abuse, which ensures the therapeutic action thereof on correct administration but does not have the action desired by the abuser when taken abusively.

This object was achieved by an abuse-resistant solid dosage form according to the invention, comprising at least one active ingredient with potential for abuse and at least one emetic spatially separate therefrom, wherein the active ingredient or active ingredients is/are present in at least one subunit (a) and the emetic is present in at least one subunit (b) and the emetic from subunit (b) does not take effect in a subject if the dosage form is correctly administered to the subject.

For the purposes of the present invention, subunits are solid formulations which in each case comprise only the active ingredient(s) or only the emetic(s) in addition to conventional auxiliary substances known to the person skilled in the art. Methods for producing corresponding subunits are known to the person skilled in the art, for example from “Coated Pharmaceutical Dosage Forms—Fundamentals, Manufacturing Techniques, Biopharmaceutical Aspects, Test Methods and Raw Materials” by Kurt H. Bauer, K. Lehmann, Hermann P. Osterwald, Rothgang, Gerhart, 1st edition, 1998, Medpharm Scientific Publishers. The corresponding description is hereby incorporated by reference and is deemed to be part of the disclosure.

The dosage form according to the invention may comprise in its respective subunits (a) and (b) in each case one or more active ingredients with potential for abuse and one or more emetics. The dosage form according to the invention preferably comprises in the corresponding subunits in each case only one active ingredient and only one emetic.

Pharmaceutical active ingredients with potential for abuse are known per se to persons skilled in the art, as are the quantities thereof to be used and processes for the production thereof, and may be present in the dosage form according to the invention as such, in the form of corresponding derivatives, in particular esters or ethers, or in each case in the form of corresponding physiologically acceptable compounds, in particular in the form of the salts or solvates thereof.

The dosage form according to the invention is particularly suitable for preventing abuse of a pharmaceutical active ingredient selected from the group consisting of opiates, opioids, tranquillizers, preferably benzodiazepines, stimulants and other narcotics. The dosage form according to the invention is particularly suitable for preventing abuse of opiates, opioids, tranquillizers, and other narcotics which are selected from the group consisting of N-{1-[2-(4-ethyl-5-oxo-2-tetrazolin-1-yl)ethyl]-4-methoxymethyl-4-piperidyl}propionanilide (alfentanil), 5,5-diallylbarbituric acid (allobarbital), allylprodine, alphaprodine, 8-chloro-1-methyl-6-phenyl-4H-[1,2,4]triazolo[4,3-a][1,4]-benzodiazepine (alprazolam), 2-diethylaminopropiophenone (amfepramone), (±)-α-methylphenethylamine (amphetamine), 2-(α-methylphenethylamino)-2-phenylacetonitrile (amphetaminil), 5-ethyl-5-isopentylbarbituric acid (amobarbital) anileridine, apocodeine, 5,5-diethylbarbituric acid (barbital), benzylmorphine, bezitramide, 7-bromo-5-(2-pyridyl)-1H-1,4-benzodiazepine-2(3H)-one (bromazepam), 2-bromo-4-(2-chlorophenyl)-9-methyl-6H-thieno[3,2-f][1,2,4]triazolo[4,3-α][1,4]diazepine (brotizolam), 17-cyclopropylmethyl-4,5α-epoxy-7α[(S)-1-hydroxy-1,2,2-trimethyl-propyl]-6-methoxy-6,14-endo-ethanomorphinan-3-ol (buprenorphine), 5-butyl-5-ethylbarbituric acid (butobarbital), butorphanol, (7-chloro-1,3-dihydro-1-methyl-2-oxo-5-phenyl-2H-1,4-benzodiazepin-3-yl) dimethylcarbamate (camazepam), (1S,2S)-2-amino-1-phenyl-1-propanol (cathine/D-norpseudoephedrine), 7-chloro-N-methyl-5-phenyl-3H-1,4-benzodiazepin-2-ylamine 4-oxide (chlordiazepoxide), 7-chloro-1-methyl-5-phenyl-1H-1,5-benzodiazepine-2,4(3H,5H)-dione (clobazam), 5-(2-chlorophenyl)-7-nitro-1H-1,4-benzodiazepin-2(3H)-one (clonazepam), clonitazene, 7-chloro-2,3-dihydro-2-oxo-5-phenyl-1H-1,4-benzodiazepine-3-carboxylic acid (clorazepate), 5-(2-chlorophenyl)-7-ethyl-1-methyl-1H-thieno[2,3-e][1,4]diazepin-2(3H)—one (clotiazepam), 10-chloro-11b-(2-chlorophenyl)-2,3,7,11b-tetrahydrooxazolo[3,2-d][1,4]benzodiazepin-6(5H)-one (cloxazolam), (−)-methyl-[3β-benzoyloxy-2β(1αH,5αH-tropane carboxylate] (cocaine), 4,5α-epoxy-3-methoxy-17-methyl-7-morphinen-6α-ol (codeine), 5-(1-cyclohexenyl)-5-ethylbarbituric acid (cyclobarbital), cyclorphan, cyprenorphine, 7-chloro-5-(2-chlorophenyl)-1H-1,4-benzodiazepin-2 (3H)-one (delorazepam), desomorphine, dextromoramide, (+)-(1-benzyl-3-dimethylamino-2-methyl-1-phenylpropyl) propionate (dextropropoxyphene), dezocine, diampromide, diamorphone, 7-chloro-1-methyl-5-phenyl-1H-1,4-benzodiazepin-2(3H)-one (diazepam), 4,5α-epoxy-3-methoxy-17-methyl-6α-morphinanol (dihydrocodeine), 4,5α-epoxy-17-methyl-3,6a-morphinandiol (dihydromorphine), dimenoxadol, dimephetamol, dimethylthiambutene, dioxaphetyl butyrate, dipipanone, (6aR,10aR)-6,6,9-trimethyl-3-pentyl-6a,7,8,10a-tetrahydro-6H-benzo[c]chromen-1-ol (dronabinol), eptazocine, 8-chloro-6-phenyl-4H-[1,2,4]triazolo[4,3-a][1,4]benzodiazepine (estazolam), ethoheptazine, ethylmethylthiambutene, ethyl[7-chloro-5-(2-fluorophenyl)-2,3-dihydro-2-oxo-1H-1,4-benzodiazepine-3-carboxylate] (ethyl loflazepate), 4,5α-epoxy-3-ethoxy-17-methyl-7-morphinen-6α-ol (ethylmorphine), etonitazene, 4,5α-epoxy-7α-(1-hydroxy-1-methylbutyl)-6-methoxy-17-methyl-6,14-endo-etheno-morphinan-3-ol (etorphine), N-ethyl-3-phenyl-8,9,10-trinorbornan-2-ylamine (fencamfamine), 7-[2-(α-methylphenethylamino)ethyl]-theophylline) (fenethylline), methylphenethylamino)propionitrile (fenproporex), N-(1-phenethyl-4-piperidyl)propionanilide (fentanyl), 7-chloro-5-(2-fluorophenyl)-1-methyl-1H-1,4-benzodiazepin-2(3H)-one (fludiazepam), 5-(2-fluorophenyl)-1-methyl-7-nitro-1H-1,4-benzodiazepin-2(3H)-one (flunitrazepam), 7-chloro-1-(2-diethylaminoethyl)-5-(2-fluorophenyl)-1H-1,4-benzodiazepin-2(3H)-one (flurazepam), 7-chloro-5-phenyl-1-(2,2,2-trifluoroethyl)-1H-1,4-benzodiazepin-2(3H)-one (halazepam), 10-bromo-11b-(2-fluorophenyl)-2,3,7,11b-tetrahydro[1,3]oxazolo[3,2-d][1,4]benzodiazepin-6(5H)-one (haloxazolam), heroin, 4,50-epoxy-3-methoxy-17-methyl-6-morphinanone (hydrocodone), 4,5α-epoxy-3-hydroxy-17-methyl-6-morphinanone (hydromorphone), hydroxypethidine, isomethadone, hydroxymethylmorphinan, 11-chloro-8,12b-dihydro-2,8-dimethyl-12b-phenyl-4H-[1,3]oxazino[3,2-d][1,4]benzodiazepine-4,7(6H)— dione (ketazolam), 1-[4-(3-hydroxyphenyl)-1-methyl-4-piperidyl]-1-propanone (ketobemidone), (3S,6S)-6-dimethylamino-4,4-diphenylheptan-3-yl acetate (levacetylmethadol (LAAM)), (−)-6-dimethylamino-4,4-diphenol-3-heptanone (levomethadone), (−)-17-methyl-3-morphinanol (levorphanol), levophenacylmorphane, lofentanil, 6-(2-chlorophenyl)-2-(4-methyl-1-piperazinylmethylene)-8-nitro-2H-imidazo[1,2-a][1,4]-benzodiazepin-1(4H)-one (loprazolam), 7-chloro-5-(2-chlorophenyl)-3-hydroxy-1H-1,4-benzodiazepin-2(3H)-one (lorazepam), 7-chloro-5-(2-chlorophenyl)-3-hydroxy-1-methyl-1H-1,4-benzodiazepin-2(3H)-one (lormetazepam), 5-(4-chlorophenyl)-2,5-dihydro-3H-imidazo[2,1-a]isoindol-5-ol (mazindol), 7-chloro-2,3-dihydro-1-methyl-5-phenyl-1H-1,4-benzodiazepine (medazepam), N-(3-chloropropyl)-α-methylphenethylamine (mefenorex), meperidine, 2-methyl-2-propyltrimethylene dicarbamate (meprobamate), meptazinol, metazocine, methylmorphine, N,α-dimethylphenethylamine (metamphetamine), (±)-6-dimethylamino-4,4-diphenol-3-heptanone (methadone), 2-methyl-3-o-tolyl-4(3H)-quinazolinone (methaqualone), methyl[2-phenyl-2-(2-piperidyl)acetate] (methylphenidate), 5-ethyl-1-methyl-5-phenylbarbituric acid (methylphenobarbital), 3,3-diethyl-5-methyl-2,4-piperidinedione (methyprylon), metopon, 8-chloro-6-(2-fluorophenyl)-1-methyl-4H-imidazo[1,5-a][1,4]benzodiazepine (midazolam), 2-(benzhydrylsulfinyl)acetamide (modafinil), 4,5α-epoxy-17-methyl-7-morphinen-3,6α-diol (morphine), myrophine, (±)-trans-3-(1,1-dimethylheptyl)-7,8,10,10α-tetrahydro-1-hydroxy-6,6-dimethyl-6H-dibenzo-[b,d]pyran-9(6αH)-one (nabilone), nalbuphene, nalorphine, narceine, nicomorphine, 1-methyl-7-nitro-5-phenyl-1H-1,4-benzodiazepin-2(3H)-one (nimetazepam), 7-nitro-5-phenyl-1H-1,4-benzodiazepin-2(3H)-one (nitrazepam), 7-chloro-5-phenyl-1H-1,4-benzodiazepin-2(3H)-one (nordazepam), norlevorphanol, 6-dimethylamino-4,4-diphenyl-3-hexanone (normethadone), normorphine, norpipanone, the exudation from plants belonging to the species Papaver somniferum (opium), 7-chloro-3-hydroxy-5-phenyl-1H-1,4-benzodiazepin-2(3H)-one (oxazepam), (cis-trans)-10-chloro-2,3,7,11b-tetrahydro-2-methyl-11b-phenyloxazolo[3,2-d][1,4]benzodiazepin-6-(5H)-one (oxazolam), 4,5α-epoxy-14-hydroxy-3-methoxy-17-methyl-6-morphinanone (oxycodone), oxymorphone, plants and parts of plants belonging to the species Papaver somniferum (including the subspecies setigerum) (Papaver somniferum), papavereturn, 2-imino-5-phenyl-4-oxazolidinone (pernoline), 1,2,3,4,5,6-hexahydro-6,11-dimethyl-3-(3-methyl-2-butenyl)-2,6-methano-3-benzazocin-8-ol (pentazocine), 5-ethyl-5-(1-methylbutyl)-barbituric acid (pentobarbital), ethyl (1-methyl-4-phenyl-4-piperidinecarboxylate) (pethidine), phenadoxone, phenomorphane, phenazocine, phenoperidine, piminodine, pholcodeine, 3-methyl-2-phenylmorpholine (phenmetrazine), 5-ethyl-5-phenylbarbituric acid (phenobarbital), α,α-dimethylphenethylamine (phentermine), 7-chloro-5-phenyl-1-(2-propynyl)-1H-1,4-benzodiazepin-2(3H)-one (pinazepam), α(2-piperidyl)benzhydryl alcohol (pipradrol), 1′-(3-cyano-3,3-diphenylpropyl)[1,4′-bipiperidine]-4′-carboxamide (piritramide), 7-chloro-1-(cyclopropylmethyl)-5-phenyl-1H-1,4-benzodiazepin-2(3H)-one (prazepam), profadol, proheptazine, promedol, properidine, propoxyphene, N-(1-methyl-2-piperidinoethyl)-N-(2-pyridyl)propionamide, methyl {3-[4-methoxycarbonyl-4-(N-phenylpropanamido)piperidino]propanoate}(remifentanil), 5-sec-butyl-5-ethylbarbituric acid (secbutabarbital), 5-allyl-5-(1-methylbutyl)-barbituric acid (secobarbital), N-{4-methoxymethyl-1-[2-(2-thienyl)ethyl]-4-piperidyl}propionanilide (sufentanil), 7-chloro-2-hydroxy-methyl-5-phenyl-1H-1,4-benzodiazepin-2(3H)-one (temazepam), 7-chloro-5-(1-cyclohexenyl)-1-methyl-1H-1,4-benzodiazepin-2(3H)-one (tetrazepam), ethyl (2-dimethylamino-1-phenyl-3-cyclohexene-1-carboxylate) (tilidine, cis and trans)), tramadol, 8-chloro-6-(2-chlorophenyl)-1-methyl-4H-[1,2,4]triazolo[4,3-a][1,4]benzodiazepine (triazolam), 5-(1-methylbutyl)-5-vinylbarbituric acid (vinylbital), (1R*,2R*)-3-(3-dimethylamino-1-ethyl-2-methyl-propyl)-phenol, (1R,2R,4S)-2-(dimethylamino)methyl-4-(p-fluorobenzyloxy)-1-(m-methoxyphenyl)cyclohexanol, in each case optionally in the form of corresponding stereoisomeric compounds and corresponding derivatives, in particular esters or ethers, and respective corresponding physiologically acceptable compounds, in particular salts and solvates.

The compounds (1R*,2R*)-3-(3-dimethylamino-1-ethyl-2-methyl-propyl)-phenol and (1R,2R,4S)-2-(dimethylamino)methyl-4-(p-fluorobenzyloxy)-1-(m-methoxyphenyl)cyclohexanol, the physiologically acceptable compounds thereof, in particular the hydrochlorides thereof and processes for the production thereof are respectively known, for example, from U.S. Pat. No. 6,248,737 (=EP693,475) and U.S. Pat. No. 5,801,201 (=EP780,369), the entire disclosures of which are incorporated herein by reference.

The dosage form according to the invention is also suitable for preventing abuse of stimulants, preferably those selected from the group consisting of amphetamine, norpseudoephedrine, methylphenidate and in each case optionally the corresponding physiologically acceptable compounds thereof, in particular the bases, salts and solvates thereof.

Suitable emetics for preventing abuse of the active ingredients are known per se to the person skilled in the art and may be present in the dosage form according to the invention as such or in the form of corresponding derivatives, in particular esters or ethers, or in each case in the form of corresponding physiologically acceptable compounds, in particular in the form of the salts or solvates thereof.

An emetic based on one or more constituents of ipecacuanha (ipecac) root, preferably based on the constituent emetine, is preferably used in the dosage form according to the invention, as are, for example, described in “Pharmazeutische Biologie—Drogen and ihre Inhaltsstoffe” by Prof. Dr. Hildebert Wagner, 2nd, revised edition, Gustav Fischer Verlag, Stuttgart, New York 1982. The corresponding literature description is hereby incorporated by reference and is deemed to be part of the disclosure.

The dosage form according to the invention preferably comprises as emetic emetine in a quantity of ≧10 mg, particularly preferably ≧20 mg and very particularly preferably ≧40 mg per dosage form, i.e. administration unit.

Apomorphine may likewise preferably be used as an emetic in the dosage form according to the invention, in particular in those which are particularly suitable for preventing parenteral or nasal abuse. If apomorphine is present in the dosage form according to the invention, the respective quantity per administration unit is preferably ≧3 mg, particularly preferably ≧5 mg and very particularly preferably ≧7 mg.

One substantial aspect of the present invention is that the emetic is practically not released into the body of a subject (i.e., is not released in an emetically effective amount) by the subunit or subunits (b) of the dosage form according to the invention when administered correctly to the subject, or is released in such small quantities that it does not have any action harmful to the subject or, on passage through the subject's body, they are only liberated in locations where they cannot be sufficiently absorbed to be effective. The emetic is preferably practically not released in the subject's body.

Those skilled in the art understand that these above-stated conditions may vary as a function of the emetic used in each case and the formulation of the subunit (b) or the dosage form. The optimum formulation for the particular emetic may be determined by simple preliminary testing.

If the dosage form according to the invention is manipulated for the purpose of abusive taking of the active ingredient, e.g. by grinding and optionally extracting the powder thus obtained with a suitable extracting agent, in addition to the active ingredient, the emetic is also obtained in a form in which it cannot easily be separated from the active ingredient, such that, on administration of the manipulated dosage form, in particular in the case of oral and/or parenteral administration, its action develops in the body and the body mounts an immune response, namely severe nausea or even vomiting, so preventing abuse of the dosage form.

The dosage form according to the invention may be formulated in a large number of ways according to conventional methods known to the person skilled in the art, wherein the subunits (a) and (b) in the dosage form according to the invention may each be present in any spatial arrangement relative to one another, provided that the above-stated conditions for release of the emetic are fulfilled. Methods for producing the dosage forms are known to the person skilled in the art, for example from “Coated Pharmaceutical Dosage Forms—Fundamentals, Manufacturing Techniques, Biopharmaceutical Aspects, Test Methods and Raw Materials” by Kurt H. Bauer, K. Lehmann, Hermann P. Osterwald, Rothgang, Gerhart, 1st edition, 1998, Medpharm Scientific Publishers. The corresponding description is hereby incorporated by reference and is deemed to be part of the disclosure.

The subunits (a) and (b) in multiparticulate form may be press-molded to form a tablet, wherein the final formulation in each case proceeds in such a manner that the subunits (a) and (b) are also retained in the resultant dosage form.

In a preferred embodiment of the present invention, subunits (a) and (b) are in each case arranged in layers relative to one another. The layered subunits (a) and (b) are preferably arranged for this purpose vertically or horizontally relative to one another in the dosage form produced according to the invention, wherein in each case one or more layered subunits (a) and one or more layered subunits (b) may be present in the dosage form, such that, apart from the preferred layer sequences (a)-(b) or (a)-(b)-(a), any desired other layer sequences may be considered.

Another preferred dosage form according to the invention is one in which subunit (b) forms a core which is completely enclosed by subunit (a), wherein an optionally swellable separation layer (c) may be present between said layers. Such a structure is preferably also suitable for the above-stated multiparticulate forms, wherein both subunits (a) and (b) and an optionally present separation layer (c) are formulated in one and the same multiparticulate form.

In a further preferred embodiment of the dosage form according to the invention, the subunit (a) forms a core, which is enclosed by subunit (b), wherein the latter comprises at least one channel which leads from the core to the surface of the dosage form.

The dosage form according to the invention may comprise, between one layer of the subunit (a) and one layer of the subunit (b), in each case one or more, preferably one, optionally swellable separation layer (c) which serves to separate subunit (a) spatially from (b). If the dosage form according to the invention comprises the layered subunits (a) and (b) and an optionally present separation layer (c) in an at least partially vertical or horizontal arrangement, the dosage form preferably assumes the form of a tablet, a coextrudate or a laminate.

In one particularly preferred embodiment, the entirety of the free surface of subunit (b) and optionally at least part of the free surface of subunit(s) (a) and optionally at least part of the free surface of the optionally present separation layer(s) (c) may be coated with at least one barrier layer (d) which prevents release of the emetic.

Another particularly preferred embodiment of the dosage form according to the invention comprises a vertical or horizontal arrangement of the layers of subunits (a) and (b) and at least one push layer (p) arranged therebetween, and optionally a separation layer (c), in which dosage form the entirety of the free surfaces of the layer structure consisting of subunits (a) and (b), the push layer (p) and the optionally present separation layer (c) are provided with a semipermeable coating (e), which is permeable to a release medium, i.e. conventionally a physiological liquid, but substantially impermeable to the active ingredient and to the emetic, and wherein this coating (e) comprises at least one opening for release of the active ingredient in the area of subunit (a).

A corresponding dosage form is known to the person skilled in the art, for example under the name oral osmotic therapeutic system (OROS), as are suitable materials and methods for the production thereof, inter alia from U.S. Pat. Nos. 4,612,008; 4,765,989 and 4,783,337, the disclosures of which are incorporated herein by reference.

In a further preferred embodiment, the subunit (a) of the dosage form according to the invention assumes the form of a tablet, the edge face and optionally one of the two main faces of which is covered with a barrier layer (d) containing the emetic.

Persons skilled in the art will understand that the auxiliary substances of the subunit(s) (a) or (b) and of the optionally present separation layer(s) (c) and/or of the barrier layer(s) (d) used in formulating the dosage form according to the invention will vary as a function of the arrangement thereof in the dosage form according to the invention, the mode of administration and as a function of the particular active ingredient and the emetic. The materials which have the requisite properties are in each case known per se to those skilled in the art.

If release of the emetic from subunit (b) of the dosage form according to the invention is prevented with the assistance of a cover, preferably a barrier layer, the subunit may consist of conventional materials known to those skilled in the art.

If a corresponding barrier layer (d) is not provided to prevent release of the emetic, the materials of the subunits should be selected such that release of the emetic from subunit (b) is virtually ruled out.

The materials which are stated below to be suitable for production of the barrier layer may preferably be used for this purpose. Preferred materials may be selected from the group consisting of alkylcelluloses hydroxyalkylcelluloses, glucans, scleroglucans, mannans, xanthans, copolymers of poly[bis(p-carboxyphenoxy)propane and sebacic acid], preferably in a molar ratio of 20:80 (marketed under the name Polifeprosan 20®), carboxymethylcelluloses, cellulose ethers, cellulose esters, nitrocelluloses, polymers based on (meth)acrylic acid and the esters thereof, polyamides, polycarbonates, polyalkylenes, polyalkylene glycols, polyalkylene oxides, polyalkylene terephthalates, polyvinyl alcohols, polyvinyl ethers, polyvinyl esters, halogenated polyvinyls, polyglycolides, polysiloxanes and polyurethanes and the copolymers thereof.

Particularly suitable materials may be selected from the group consisting of methylcellulose, ethylcellulose, hydroxypropylcellulose, hydroxypropylmethylcellulose, hydroxybutylmethylcellulose, cellulose acetate, cellulose propionate (of low, medium or high molecular weight), cellulose acetate propionate, cellulose acetate butyrate, cellulose acetate phthalate, carboxymethylcellulose, cellulose triacetate, sodium cellulose sulfate, polymethyl methacrylate, polyethyl methacrylate, polybutyl methacrylate, polyisobutyl methacrylate, polyhexyl methacrylate, polyisodecyl methacrylate, polylauryl methacrylate, polyphenyl methacrylate, polymethyl acrylate, polyisopropyl acrylate, polyisobutyl acrylate, polyoctatdecyl acrylate, polyethylene, low density polyethylene, high density polyethylene, polypropylene, polyethylene glycol, polyethylene oxide, polyethylene terephthalate, polyvinyl alcohol, polyvinyl isobutyl ether, polyvinyl acetate and polyvinyl chloride.

Particularly suitable copolymers may be selected from the group comprising copolymers of butyl methacrylate and isobutyl methacrylate, copolymers of methyl vinyl ether and maleic acid of high molecular weight, copolymers of methyl vinyl ether and maleic acid monoethyl ester, copolymers of methyl vinyl ether and maleic anhydride and copolymers of vinyl alcohol and vinyl acetate.

Further biodegradable materials which are particularly suitable for formulating the barrier layer include starch-filled polycaprolactone [WO98/20073], aliphatic polyesteramides [U.S. Pat. No. 6,344,535 (=DE 19 753 534); CA 2,317,747 (=DE 19 800 698); U.S. Pat. No. 5,928,739 (=EP 820,698)], aliphatic and aromatic polyester urethanes [U.S. Pat. No. 6,821,588 (=DE 198 22 979)], polyhydroxyalkanoates, in particular polyhydroxybutyrates, polyhydroxyvalerates, casein [U.S. Pat. No. 5,681,517 (=DE 43 09 528)], polylactides and copolylactides [U.S. Pat. No. 6,235,825 (=EP 980,894)], the disclosures of which are incorporated by reference herein.

The above-stated materials may optionally be blended with further conventional auxiliary substances known to those skilled in the art, preferably selected from the group consisting of glyceryl monostearate, semi-synthetic triglyceride derivatives, semi-synthetic glycerides, hydrogenated castor oil, glyceryl palmitostearate, glyceryl behenate, polyvinylpyrrolidone, gelatine, magnesium stearate, stearic acid, sodium stearate, talcum, sodium benzoate, boric acid and colloidal silica, fatty acids, substituted triglycerides, glycerides, polyoxyalkylene glycols and the derivatives thereof.

If the dosage form according to the invention comprises a separation layer (c), said layer, like the subunit (b) not covered by a barrier layer, may preferably consist of the above-stated materials described for the barrier layer. The person skilled in the art will understand that release of the emetic from the particular subunit may be controlled by the thickness of the separation layer.

The dosage form according to the invention for oral administration of one or more active ingredients is particularly suitable for preventing oral, nasal and/or parenteral abuse of such active ingredients.

One or more active ingredients at least partially in delayed-release form may also be present, wherein delayed release may be achieved with the assistance of conventional materials and methods known to the person skilled in the art, for example by embedding the active ingredient in a delayed-release matrix or by the application of one or more delayed-release coatings. Active ingredient release must, however, be controlled such that, in the event of correct administration of the dosage form, the active ingredient or active ingredients are virtually completely released before the emetic can exert an impairing effect.

If the dosage form according to the invention is intended for oral administration, it may also preferably comprise a coating which is resistant to gastric juices and dissolves as a function of the pH value of the release environment. By means of this coating, it is possible to ensure that the dosage form according to the invention passes through the stomach undissolved and the active ingredient is only released in the intestines. The coating which is resistant to gastric juices preferably dissolves at a pH value of between 5 and 7.5. The subunit (b) should then preferably be formulated such that the emetic is practically not released in the body.

Corresponding materials and methods for the controlled release of active ingredients and for the application of coatings which are resistant to gastric juices are known to the person skilled in the art, for example from “Coated Pharmaceutical Dosage Forms—Fundamentals, Manufacturing Techniques, Biopharmaceutical Aspects, Test Methods and Raw Materials” by Kurt H. Bauer, K. Lehmann, Hermann P. Osterwald, Rothgang, Gerhart, 1st edition, 1998, Medpharm Scientific Publishers. The corresponding literature description is hereby introduced as a reference and is deemed to be part of the disclosure.

In a further preferred embodiment, the quantity of the emetic in the dosage form according to the invention is selected such that, in the event of correct oral administration, no negative action is caused. If, however, the intended dosage of the dosage form is exceeded inadvertently, in particular by children, or in the event of abuse, nausea or an inclination to vomit are produced. The particular quantity of the emetic which can still be tolerated by the patient in the event of correct oral administration may be determined by the person skilled in the art by simple preliminary testing.

The dosage forms according to the invention have the advantage that they are protected against nasal and/or parenteral and optionally also against oral abuse, without the risk of harm to the patient being treated or a reduction in efficacy of the respective active ingredient when administered correctly. They may be produced simply and comparatively economically.

The invention is explained in further detail below with reference to illustrative Examples. These explanations are given merely by way of example and do not limit the overall scope of the invention.

### EXAMPLES

The quantities indicated below relate in each case to an individual dosage form. A batch from a single production run comprised 1000 dosage forms.

### Example 1

### Jacketed Tablets

Core

| | |
|---|---|
| Emetine | 50 mg |
| Hydrogenated castor oil (Cutina HR) | 50 mg |

Emetine and finely powdered hydrogenated castor oil were mixed and press-molded in a tablet press to form round, biconvex tables of a diameter of 6.5 mm.

Jacket

| | |
|---|---|
| Morphine sulfate pentahydrate | 60 mg |
| Methylhydroxypropylcellulose 100,000 mPa · s | 100 mg |
| (Metolose 90 SH 100,000, ShinEtsu) | |
| Microcrystalline cellulose (Avicel PH 102) | 165 mg |
| Lactose monohydrate | 165 mg |
| Magnesium stearate | 5 mg |
| Colloidal silicon dioxide | 5 mg |

All the jacket constituents were mixed; approx. 250 mg of the mixture were placed in the tablet die in a tablet press with a tool for 13 mm biconvex tablets, the 6.5 mm core was inserted centrally, the remaining 250 mg of jacket mixture were added and the jacket was pressed around the core.

### Example 2

### Jacketed Tablets

Core

| | |
|---|---|
| Emetine | 50 mg |
| Hydrogenated castor oil (Cutina HR) | 50 mg |

Emetine and finely powdered hydrogenated castor oil were mixed and press-molded in a tablet press to form round, biconvex tablets of a diameter of 6.5 mm.

Jacket

| | |
|---|---|
| Oxycodone hydrochloride | 30 mg |
| Spray-dried lactose | 300 mg |
| Eudragit RSPM | 70 mg |
| Stearyl alcohol | 115 mg |
| Magnesium stearate | 5 mg |
| Talcum | 10 mg |

Oxycodone hydrochloride, spray-dried lactose and Eudragit RSPM were intimately mixed together for approx. 5 min in a suitable mixer. During mixing, the mixture was granulated with such a quantity of purified water that a moist, granulated mass was formed. The resultant granular product was dried in a fluidized bed at 60° C. and passed through a 2.5 mm screen. The granular product was then dried again as described above and passed through a 1.5 mm screen. The stearyl alcohol was melted at 60-70° C. and added to the granular product in a mixer. After cooling, the mass was passed through 1.5 mm screen. From the resultant granular product, approx. 265 mg of the mixture were placed in the tablet die in a tablet press with a tool for 13 mm biconvex tablets, the 6.5 mm core was inserted centrally, the remaining 265 mg of the jacket mixture were added and the jacket was pressed around the core.

### Example 3

### Jacketed Tablets

Core

| | |
|---|---|
| Emetine | 50 mg |
| Spray-dried lactose | 46 mg |
| Magnesium stearate | 2 mg |
| Colloidal silicon dioxide | 2 mg |

All the constituents were mixed and press-molded in a tablet press to form round, biconvex tablets of a diameter of 6.5 mm.

Coating on core

| | |
|---|---|
| Cellulose acetate with 39.8% acetate | 9.5 mg |
| Macrogol 3350 | 0.5 mg |

The coating constituents were dissolved in an acetone-water mixture (95:5 parts by weight) and sprayed onto the cores.

Jacket

| | |
|---|---|
| Morphine sulfate pentahydrate | 60 mg |
| Methylhydroxypropylcellulose 100,000 mPa · s | 100 mg |
| (Metolose 90 SH 100,000, ShinEtsu) | |
| Microcrystalline cellulose (Avicel PH 102) | 165 mg |
| Lactose monohydrate | 165 mg |
| Magnesium stearate | 5 mg |
| Colloidal silicon dioxide | 5 mg |

All the jacket constituents were mixed; approx. 250 mg of the mixture were placed in the tablet die in a tablet press with a tool for 13 mm biconvex tablets, the core coated with cellulose acetate was inserted centrally, the remaining 250 mg of jacket mixture were added and the jacket was pressed around the core.

### Example 4

### Jacketed Tablets

Core

| | |
|---|---|
| Emetine hydrochloride pentahydrate | 60 mg |
| Hydrogenated castor oil (Cutina HR) | 40 mg |

Emetine hydrochloride pentahydrate and finely powdered hydrogenated castor oil were mixed and press-molded in a tablet press to form round, biconvex tables of a diameter of 6.5 mm.

Coating on core

| | |
|---|---|
| Cellulose acetate with 39.8% acetate | 9.5 mg |
| Macrogol 3350 | 0.5 mg |

The coating constituents were dissolved as a 3.8% solution in an acetone-water mixture (95:5 parts by weight) and sprayed onto the cores.

Jacket

| | |
|---|---|
| Morphine sulfate pentahydrate | 60 mg |
| Methylhydroxypropylcellulose 100,000 mPa · s | 100 mg |
| (Metolose 90 SH 100,000, ShinEtsu) | |
| Microcrystalline cellulose (Avicel PH 102) | 165 mg |
| Lactose monohydrate | 165 mg |
| Magnesium stearate | 5 mg |
| Colloidal silicon dioxide | 5 mg |

All the jacket constituents were mixed; approx. 250 mg of the mixture were placed in the tablet die in a tablet press with a tool for 13 mm biconvex tablets, the 6.5 mm core coated with cellulose acetate was inserted centrally, the remaining 250 mg of jacket mixture were added and the jacket was pressed around the core.

### Example 5

### Oral Osmotic Therapeutic System (OROS)

Active ingredient layer

| | |
|---|---|
| Morphine sulfate pentahydrate | 125 mg |
| Macrogol 200,000 | 280 mg |
| Povidone (MW_{N} 40,000) | 26 mg |
| Magnesium stearate | 4 mg |

The morphine sulfate and macrogol were dry-mixed in a planetary mixer and then converted into a moist mass by slow addition of a solution of the povidone in 115 mg of ethanol and the mass was then pressed through a 0.8 mm screen. After 24 hours' drying at room temperature in a fume hood, the particles were pressed together with the magnesium stearate through a 1.0 mm screen and mixed in a container mixer.

Push layer

| | |
|---|---|
| Methylhydroxypropylcellulose 6 mPa · s | 13 mg |
| Sodium chloride | 80 mg |
| Macrogol 7,000,000 | 166 mg |
| Magnesium stearate | 1 mg |

The sodium chloride, macrogol and half the methylhydroxypropylcellulose were dry-mixed for 3 minutes in a fluidized bed granulator and then granulated and dried by spraying on a solution of the second half of the methylhydroxypropylcellulose in 75 mg with introduction of hot air. The granular product was then pressed together with the magnesium stearate through a 2.5 mm screen in a Comil.

Emetic layer

| | |
|---|---|
| Emetine | 50 mg |
| Hydrogenated castor oil (Cutina HR) | 50 mg |

Emetine and hydrogenated castor oil were precompressed in a tablet press with a 10 mm precompression punch to form approx. 250 mg compression moldings. These preliminary compression moldings were then comminuted by means of a crusher and a 1.0 mm screen.

### Production of the Three Layer Tablets

For each tablet, 100 mg of the granular product for the emetic layer, 260 mg of the push layer and 435 mg of the active ingredient layer were introduced in succession into the die of a suitable tablet press and press-molded to form a three layer tablet.

Coating on core

| | |
|---|---|
| Cellulose acetate with 39.8% acetate | 38 mg |
| Macrogol 3350 | 2 mg |

The coating constituents were dissolved as a 3.8% solution in an acetone-water mixture (95:5 parts by weight) and sprayed onto the cores. Two 0.75 mm holes were drilled through the coating in order to connect the active ingredient layer with external environment of the system.

### Example 6

### Oral Osmotic Therapeutic System

Production proceeded in a manner similar to Example 6, except that the emetic layer was of the following composition:

| | |
|---|---|
| Emetine hydrochloride pentahydrate | 60 mg |
| Hydrogenated castor oil (Cutina HR) | 40 mg |

Emetine hydrochloride pentahydrate and hydrogenated castor oil were precompressed in a tablet press with a 10 mm precompression punch to form approx. 250 mg compression moldings. The preliminary compression moldings were then comminuted by means of a crusher and a 1.0 mm screen. All the other production steps proceeded as explained in Example 6.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. An abuse-resistant solid dosage form containing at least one active ingredient with potential for abuse and at least one emetic spatially separate from said at least one active ingredient; wherein:
the at least one active ingredient is present in at least one subunit (a) of said body;
the at least one emetic is present in at least one subunit (b) of said body;
the subunit (b) forms a core which is completely enclosed by a layer composed of subunit (a);
at least one optionally swellable separation layer is arranged between the subunits (a) and (b); and
the emetic from subunit (b) is not released in an emetically effective amount in a subject when the dosage form is correctly administered to said subject.

2. A dosage form according to claim 1, wherein said at least one active ingredient with potential for abuse is selected from the group consisting of opiates, opioids, tranquilizers, stimulants and narcotics.

3. A dosage form according to claim 2, wherein said at least one active ingredient is a benzodiazepine tranquilizer.

4. A dosage form according to claim 2, wherein said at least one active ingredient with potential for abuse is selected from the group consisting of: N-{1-[2-(4-ethyl-5-oxo-2-tetrazolin-1-yl)ethyl]-4-methoxymethyl-4-piperidyl}propionanilide (alfentanil), 5,5-diallylbarbituric acid (allobarbital), allylprodine, alphaprodine, 8-chloro-1-methyl-6-phenyl-4H-[1,2,4]triazolo[4,3-a][1,4]-benzodiazepine (alprazolam), 2-diethylaminopropiophenone (amfepramone), (±)-α-methylphenethylamine (amphetamine), 2-(α-methylphenethylamino)-2-phenylacetonitrile (amphetaminil), 5-ethyl-5-isopentylbarbituric acid (amobarbital) anileridine, apocodeine, 5,5-diethylbarbituric acid (barbital), benzylmorphine, bezitramide, 7-bromo-5-(2-pyridyl)-1H-1,4-benzodiazepine-2(3H)-one (bromazepam), 2-bromo-4-(2-chlorophenyl)-9-methyl-6H-thieno[3,2-f][1,2,4]triazolo[4,3-a][1,4]diazepine (brotizolam), 17-cyclopropylmethyl-4,5α-epoxy-7α[(S)-1-hydroxy-1,2,2-trimethyl-propyl]-6-methoxy-6,14-endo-ethanomorphinan-3-ol (buprenorphine), 5-butyl-5-ethylbarbituric acid (butobarbital), butorphanol, (7-chloro-1,3-dihydro-1-methyl-2-oxo-5-phenyl-2H-1,4-benzodiazepin-3-yl) dimethylcarbamate (camazepam), (1S,2S)-2-amino-1-phenyl-1-propanol (cathine/D-norpseudoephedrine), 7-chloro-N-methyl-5-phenyl-3H-1,4-benzodiazepin-2-ylamine 4-oxide (chlordiazepoxide), 7-chloro-1-methyl-5-phenyl-1H-1,5-benzodiazepine-2,4(3H,5H)-dione (clobazam), 5-(2-chlorophenyl)-7-nitro-1H-1,4-benzodiazepin-2(3H)-one (clonazepam), clonitazene, 7-chloro-2,3-dihydro-2-oxo-5-phenyl-1H-1,4-benzodiazepine-3-carboxylic acid (clorazepate), 5-(2-chlorophenyl)-7-ethyl-1-methyl-1H-thieno[2,3-e][1,4]diazepin-2(3H)-one (clotiazepam), 10-chloro-11b-(2-chlorophenyl)-2,3,7,11b-tetrahydrooxazolo[3,2-d][1,4]benzodiazepin-6(5H)-one (cloxazolam), (−)-methyl-[3β-benzoyloxy-2β(1αH,5αH)-tropane carboxylate] (cocaine), 4,5α-epoxy-3-methoxy-17-methyl-7-morphinen-6α-ol (codeine), 5-(1-cyclohexenyl)-5-ethylbarbituric acid (cyclobarbital), cyclorphan, cyprenorphine, 7-chloro-5-(2-chlorophenyl)-1H-1,4-benzodiazepin-2(3H)-one (delorazepam), desomorphine, dextromoramide, (+)-(1-benzyl-3-dimethylamino-2-methyl-1-phenylpropyl) propionate (dextropropoxyphene), dezocine, diampromide, diamorphone, 7-chloro-1-methyl-5-phenyl-1H-1,4-benzodiazepin-2(3H)-one (diazepam), 4,5α-epoxy-3-methoxy-17-methyl-6α-morphinanol (dihydrocodeine), 4,5α-epoxy-17-methyl-3,6a-morphinandiol (dihydromorphine), dimenoxadol, dimephetamol, dimethylthiambutene, dioxaphetyl butyrate, dipipanone, (6aR,10aR)-6,6,9-trimethyl-3-pentyl-6a,7,8,10a-tetrahydro-6H-benzo[c]chromen-1-ol (dronabinol), eptazocine, 8-chloro-6-phenyl-4H-[1,2,4]triazolo[4,3-a][1,4]benzodiazepine (estazolam), ethoheptazine, ethylmethylthiambutene, ethyl[7-chloro-5-(2-fluorophenyl)-2,3-dihydro-2-oxo-1H-1,4-benzodiazepine-3-carboxylate] (ethyl loflazepate), 4,5α-epoxy-3-ethoxy-17-methyl-7-morphinen-6α-ol (ethylmorphine), etonitazene, 4,5α-epoxy-7α-(1-hydroxy-1-methylbutyl)-6-methoxy-17-methyl-6,14-endo-etheno-morphinan-3-ol (etorphine), N-ethyl-3-phenyl-8,9,10-trinorbornan-2-ylamine (fencamfamine), 7-[2-(α-methylphenethylamino)ethyl]-theophylline) (fenethylline), 3-α-methylphenethylamino)propionitrile (fenproporex), N-(1-phenethyl-4-piperidyl)propionanilide (fentanyl), 7-chloro-5-(2-fluorophenyl)-1-methyl-1H-1,4-benzodiazepin-2(3H)-one (fludiazepam), 5-(2-fluorophenyl)-1-methyl-7-nitro-1H-1,4-benzodiazepin-2(3H)-one (flunitrazepam), 7-chloro-1-(2-diethylaminoethyl)-5-(2-fluorophenyl)-1H-1,4-benzodiazepin-2(3H)-one (flurazepam), 7-chloro-5-phenyl-1-(2,2,2-trifluoroethyl)-1H-1,4-benzodiazepin-2(3H)-one (halazepam), 10-bromo-11b-(2-fluorophenyl)-2,3,7,11b-tetrahydro[1,3]oxazolo[3,2-d][1,4]benzodiazepin-6(5H)-one (haloxazolam), heroin, 4,5α-epoxy-3-methoxy-17-methyl-6-morphinanone (hydrocodone), 4,5α-epoxy-3-hydroxy-17-methyl-6-morphinanone (hydromorphone), hydroxypethidine, isomethadone, hydroxymethylmorphinan, 11-chloro-8,12b-dihydro-2,8-dimethyl-12b-phenyl-4H-[1,3]oxazino[3,2-d][1,4]benzodiazepine-4,7(6H)-dione (ketazolam), 1-[4-(3-hydroxyphenyl)-1-methyl-4-piperidyl]-1-propanone (ketobemidone), (3S,6S)-6-dimethylamino-4,4-diphenylheptan-3-yl acetate (levacetylmethadol (LAAM)), (−)-6-dimethylamino-4,4-diphenol-3-heptanone (levomethadone), (−)-17-methyl-3-morphinanol (levorphanol), levophenacylmorphane, lofentanil, 6-(2-chlorophenyl)-2-(4-methyl-1-piperazinylmethylene)-8-nitro-2H-imidazo[1,2-a][1,4]-benzodiazepin-1(4H)-one (loprazolam), 7-chloro-5-(2-chlorophenyl)-3-hydroxy-1H-1,4-benzodiazepin-2(3H)-one (lorazepam), 7-chloro-5-(2-chlorophenyl)-3-hydroxy-1-methyl-1H-1,4-benzodiazepin-2(3H)-one (lormetazepam), 5-(4-chlorophenyl)-2,5-dihydro-3H-imidazo[2,1-a]isoindol-5-ol (mazindol), 7-chloro-2,3-dihydro-1-methyl-5-phenyl-1H-1,4-benzodiazepine (medazepam), N-(3-chloropropyl)-α-methylphenethylamine (mefenorex), meperidine, 2-methyl-2-propyltrimethylene dicarbamate (meprobamate), meptazinol, metazocine, methylmorphine, N,α-dimethylphenethylamine (metamphetamine), (±)-6-dimethylamino-4,4-diphenol-3-heptanone (methadone), 2-methyl-3-o-tolyl-4(3H)-quinazolinone (methaqualone), [2-phenyl-2-(2-piperidyl)acetate] (methylphenidate), 5-ethyl-1-methyl-5-phenylbarbituric acid (methylphenobarbital), 3,3-diethyl-5-methyl-2,4-piperidinedione (methyprylon), metopon, 8-chloro-6-(2-fluorophenyl)-1-methyl-4H-imidazo[1,5-a][1,4]benzodiazepine (midazolam), 2-(benzhydrylsulfinyl)acetamide (modafinil), 4,5α-epoxy-17-methyl-7-morphinen-3,6α-diol (morphine), myrophine, (±)-trans-3-(1,1-dimethylheptyl)-7,8,10,10a-tetrahydro-1-hydroxy-6,6-dimethyl-6H-dibenzo-[b,d]pyran-9(6αH)-one (nabilone), nalbuphene, nalorphine, narceine, nicomorphine, 1-methyl-7-nitro-5-phenyl-1H-1,4-benzodiazepin-2(3H)-one (nimetazepam), 7-nitro-5-phenyl-1H-1,4-benzodiazepin-2(3H)-one (nitrazepam), 7-chloro-5-phenyl-1H-1,4-benzodiazepin-2(3H)-one (nordazepam), norlevorphanol, 6-dimethylamino-4,4-diphenyl-3-hexanone (normethadone), normorphine, norpipanone, the exudation from plants belonging to the species Papaver somniferum (opium), 7-chloro-3-hydroxy-5-phenyl-1H-1,4-benzodiazepin-2(3H)-one (oxazepam), (cis-trans)-10-chloro-2,3,7,11b-tetrahydro-2-methyl-11b-phenyloxazolo[3,2-d][1,4]benzodiazepin-6-(5H)-one (oxazolam), 4,5α-epoxy-14-hydroxy-3-methoxy-17-methyl-6-morphinanone (oxycodone), oxymorphone, plants and parts of plants belonging to the species Papaver somniferum (including the subspecies setigerum) (Papaver somniferum), papavereturn, 2-imino-5-phenyl-4-oxazolidinone (pernoline), 1,2,3,4,5,6-hexahydro-6,11-dimethyl-3-(3-methyl-2-butenyl)-2,6-methano-3-benzazocin-8-ol (pentazocine), 5-ethyl-5-(1-methylbutyl)-barbituric acid (pentobarbital), ethyl (1-methyl-4-phenyl-4-piperidinecarboxylate) (pethidine), phenadoxone, phenomorphane, phenazocine, phenoperidine, piminodine, pholcodeine, 3-methyl-2-phenylmorpholine (phenmetrazine), 5-ethyl-5-phenylbarbituric acid (phenobarbital), α,α-dimethylphenethylamine (phentermine), 7-chloro-5-phenyl-1-(2-propynyl)-1H-1,4-benzodiazepin-2(3H)-one (pinazepam), α(2-piperidyl)benzhydryl alcohol (pipradrol), 1′-(3-cyano-3,3-diphenylpropyl)[1,4′-bipiperidine]-4′-carboxamide (piritramide), 7-chloro-1-(cyclopropylmethyl)-5-phenyl-1H-1,4-benzodiazepin-2(3H)-one (prazepam), profadol, proheptazine, promedol, properidine, propoxyphene, N-(1-methyl-2-piperidinoethyl)-N-(2-pyridyl)propionamide, methyl {3-[4-methoxycarbonyl-4-(N-phenylpropanamido)piperidino]propanoate}(remifentanil), 5-sec-butyl-5-ethylbarbituric acid (secbutabarbital), 5-allyl-5-(1-methylbutyl)-barbituric acid (secobarbital), N-{4-methoxymethyl-1-[2-(2-thienyl)ethyl]-4-piperidyl}propionanilide (sufentanil), 7-chloro-2-hydroxy-methyl-5-phenyl-1H-1,4-benzodiazepin-2(3H)-one (temazepam), 7-chloro-5-(1-cyclohexenyl)-1-methyl-1H-1,4-benzodiazepin-2(3H)-one (tetrazepam), ethyl (2-dimethylamino-1-phenyl-3-cyclohexene-1-carboxylate) (tilidine, cis and trans)), tramadol, 8-chloro-6-(2-chlorophenyl)-1-methyl-4H-[1,2,4]triazolo[4,3-a][1,4]benzodiazepine (triazolam), 5-(1-methylbutyl)-5-vinylbarbituric acid (vinylbital), (1R*,2R*)-3-(3-dimethylamino-1-ethyl-2-methyl-propyl)-phenol, (1R,2R,4S)-2-(dimethylamino)methyl-4-(p-fluorobenzyloxy)-1-(m-methoxyphenyl)cyclohexanol, and corresponding esters, ethers, pharmaceutically acceptable salts and solvates thereof.

5. A dosage form according to claim 2, wherein said at least one active ingredient with potential for abuse is a stimulant selected from the group consisting of amphetamine, norpseudoephedrine, methylphenidate or a salt or solvate of any of the foregoing.

6. A dosage form according to claim 1, wherein said at least one emetic comprises apomorphine or at least one constituent of ipecacuanha root.

7. A dosage form according to claim 6, wherein said at least one ememtic comprises emetine.

8. A dosage form according to claim 1, having the form of a tablet.

9. A dosage form according to claim 1, wherein the entirety of subunit (b) is coated with at least one barrier layer which hinders release of the emetic.

10. A dosage form according to claim 9, wherein said barrier layer further coats at least part of the free surface of subunit (a).

11. A dosage form according to claim 9, wherein said barrier layer further coats at least part of the surface of the separation layer.

12. A dosage form according to claim 1, further comprising a push layer between the layers of subunits (a) and (b), and all free surfaces of the layer structure comprising subunits (a) and (b) and the push layer are provided with a semi-permeable coating, which is permeable to a release medium but is substantially impermeable to the active ingredient and the emetic, and wherein said semi-permeable coating in the area of subunits (a) comprises at least one opening for release of the active ingredient.

13. A dosage form according to claim 1, wherein at least one active ingredient is at least partially present in a delayed-release form.

14. A dosage form according to claim 1, wherein said dosage form is orally administrable.

15. A dosage form according to claim 13, further comprising at least one exterior coating resistant to gastric juices.
